(19) United States
(12) Patent Application Publication
(10) Pub. No.: US 2026/0083378 A1
(43) Pub. Date: Mar. 26, 2026

(54) DC COUPLED ELECTROCARDIOGRAM ANALOG FRONT END

(71) Applicant: Bardy Diagnostics, Inc., Bellevue, WA (US)

(72) Inventor: Yunqiang LIN, Bellevue, WA (US)

(21) Appl. No.: 19/337,058

(22) Filed: Sep. 23, 2025

Related U.S. Application Data
(60) Provisional application No. 63/698,752, filed on Sep. 25, 2024.

(51) Int. Cl. A61B 5/308; A61B 5/256; A61B 5/28; A61B 5/305

(52) U.S. Cl. CPC A61B 5/308; A61B 5/256; A61B 5/28; A61B 5/305; A61B 2560/0214; A61B 2560/0468

(57) ABSTRACT

A Direct Current (DC) coupled analog front end for an electrocardiogram (ECG) measuring device is provided that includes an amplifying stage including a first op-amp that is configured to receive a signal input from an ECG patch at a first input port and a feedback signal at a second input port and to produce an amplified output of an ECG signal via a first output port; a feedback stage including a second op-amp that is configured to receive the amplified output and produce the feedback signal at a second output port; and a DC source, coupled between the signal input from the ECG patch and the first input port.

### CROSS-REFERENCES TO RELATED DISCLOSURES

The present disclosure claims priority to U.S. Provisional Patent Application 63/698,752, filed on Sep. 25, 2024, titled “DC Coupled Electrocardiogram Analog Front End”, which is incorporated herein in its entirety.

### FIELD

The present disclosure generally relates to medical tools, and in particular to an analog front end (AFE) for use in electrocardiogram (ECG) signal gathering.

### BACKGROUND

Electrocardiograms (ECGs, also referred to as EKGs) are recordings of the electrical signals of a biological subject's heart. Electrocardiography measures the electrical activity of the cardiac cycle; graphing the voltage over time due to the muscle depolarization and repolarization that occurs during a heartbeat. Various medical conditions can be detected through the analysis of ECGs to identify shifts in rhythm, magnitude, or waveform shape from a baseline for the biological subject. An ECG in a human biological subject is measured from a baseline voltage and includes a series of waves (e.g., P-wave, Q-wave) that can be used to diagnose various cardiac conditions based on the relative magnitudes and timings in one heartbeat or across (whether consistently or sporadically) across several heartbeats.

### SUMMARY

The present disclosure provides enhanced Direct Current (DC) coupled electrocardiogram (ECG) analog front end (AFE). An ECG measurement patch adhered to the skin for the longitudinal analysis of a biological subject's ECG over an extended duration (e.g., a month or more) needs to be powered over the length of analysis period, which may require the replacement, recharging, or conservation of battery power in the device. As these devices are adhered to the skin of the biological subject, design goals frequently prioritize reducing power consumption, which reduces the size and weight of the needed battery and avoid the need to replace or recharge the battery, which in turn reduces the risk of an operator forgetting to replace/recharge the battery, improves adhesion to the biological subject's skin, reduces discomfort of wearing the devices, and various other benefits. However, conventional ECG AFEs often make sacrifices in one or more design goals, which may also include the responsiveness, accuracy, and cost of the devices used therein. Accordingly, conventional implementations offer suffer from one or more of high-power consumption, differential thermal noise, high noise (e.g., due to difference potential of the right-leg-drive (RLD) and the reference of the input stage, and complex designs with associated high costs and large sizes. Accordingly, the described devices are smaller and lighter and have a power source that can last over a longer duration that previous devices, among other benefits offered.

The design discussed in the present disclosure offers DC coupling at equal or lower power, larger input dynamic range, and faster transient response compared to conventional ECG AFEs.

For example, the design described in the present disclosure may draw less power than conventional designs, drawing approximately 20-25 microamps (μA) compared to conventional current draws of 40 μA or more today, thereby providing a 30% longer data recording capability for same battery capacity and system capabilities or the freed power budget can be used toward lower noise op-amp, higher power precision op amp to further lower system noise. Additionally or alternatively, a lower power consumption allows use of lower-capacity batteries. A smaller battery may also be used to produce a next-generation ECG monitor which is smaller and lighter, which can potentially increase patient comfort and increase the adhesion time of the patch, given a reduced force on the patient adhesive obtained from a smaller system mass.

For example, the design described in the present disclosure have experimentally proven to have single digit microvolt peak-to-peak (μVpp) level noise without low noise precision op-amp. It is feasible to optimize the design to lower single digits (e.g., 3-6 μVpp) with the inclusion of precision op amps in the design. Comparatively, conventional designs offer 11-15 μVpp noise levels. Thus, this lower noise may lead to an enhanced ECG signal, easier to detect arrythmias, improved algorithm performance and consistency, describe a clearer P-wave, or detection of smaller amplitude P-waves.

For example, the design described in the present disclosure provides a higher impedance at wider frequency bands than conventional designs (e.g., greater than 10 Gigaohms (GΩ) versus the conventional less than 5 Megaohms (MΩ) for frequencies between 0-200 Hertz (Hz)). This feature provides more consistent ECG match across patients with different skin contact resistance, which can reduce recording invalid/noisy data before the skin-adhesive-electrode impedance has settled (which can take between tens of minutes to up to a day). This ensures near-real time monitoring and reduce the impact from improper skin preparation and additional valid data collection during the settling time.

For example, the design described in the present disclosure offers a faster transient response than conventional designs, providing responses in the order of milliseconds for 0.1 V DC shifts. This can produce better/clearer ECG tracing and faster response to baseline wander and could potentially eliminate the need of backend post processing.

The present design uses a first stage with an op-amp (e.g., an amplifying stage) that may be used with various second stages that use one or more op-amps to provide feedback to the first stage (e.g., a feedback stage). The first stage includes a lowpass filter (LPF), a shunting path for the op-amp, and a LPF around the op-amp.

In some embodiments, the first op-amp circuit is configured with a non-inverting op-amp, and may have a gain of 330, and a passband of 1-45 Hertz (Hz). The second op-amp circuitry serves as common mode drive and direct current (DC) cancelation, and may be configured as integrator with 0 decibels (dB) at a frequency of 0.007 Hz. This pole together with the first op-amp forms a closed-loop feedback circuit, which results in a high pass filter of 0.007 Hz. The two op-amps interface with two electrodes respectively. The first op-amp is configured as noninverting with DC coupling from the first electrode. The second op-amp is configured as integrator using the first op-amp's output as its input, and drive to the second electrode. Before the first op-amp, a passive lowpass filter consists of protection resistor and shunting capacitor. This arrangement takes the advantage of protection resistor to filter high frequency content (e.g., ˜9 kHz or higher). After the two op-amp-based closed loop, an additional passive anti-aliasing filter can be added at the trade-off of extra power consumption.

In some embodiments, the first op-amp can be configured with capacitive feedback instead of resistive feedback. Capacitive feedback offers better passband flatness at the trade-off of corner frequency accuracy. Because of better granularity of resistors available, resistive feedback offers better performance adjusting and component flexibility than capacitive feedback.

In some embodiments, a third op-amp configured as inverter unity gain can be added to invert the second op-amp's output and apply that inverted output to the first op-amp's feedback reference. This arrangement significantly improves the transient response at the trade-off of the additional op-amp circuitry.

In some embodiments, the second op-amp can be split into two op-amp-based circuitries: a first that serves a unity gain common mode drive, a second that cancels the DC offset. This arrangement improves the transient response, reduces the sensitivity to skin-electrode impedance, at the trade-off of the additional op-amp circuitry.

Each of the described embodiments may add the capability of sensing the feedback of the second op-amp. This additional sensing allows aggressive transient response, wider dynamic range at low frequency, at the trade-off of additional ADC channel data storage.

In one embodiment, a Direct Current (DC) coupled analog front end for an electrocardiogram (ECG) measuring device is provided, comprising: an amplifying stage including a first op-amp that is configured to receive a signal input from an ECG patch at a first input port and a feedback signal at a second input port and to produce an amplified output of an ECG signal via a first output port; a feedback stage including a second op-amp that is configured to receive the amplified output and produce the feedback signal at a second output port; and a DC source, coupled between the signal input from the ECG patch and the first input port.

In some such embodiments, the DC coupled analog front end further comprises a passive lowpass filter in communication between the first output port of the first op-amp and a signal output for an ECG signal.

In some such embodiments, the second op-amp is an inverting op-amp that receives the amplified output at an inverting input port and a common voltage at a feedback input port to produce the feedback signal, wherein an input resistor is disposed between the inverting input port and a first output port of the first op-amp and an input capacitor is disposed between the inverting input port and the second output port.

In some such embodiments, the DC coupled analog front end further comprises a third op-amp disposed in the feedback stage, and configured to produce a second feedback signal that the first op-amp is configured to receive at the second input port with the feedback signal produced by the second op-amp received at the first input port of the first op-amp, the third op-amp receiving the feedback signal at a second inverting import port and the common voltage at a second feedback input port to produce the second feedback signal.

In some such embodiments, the DC coupled analog front end further comprises a third op-amp disposed in the feedback stage, wherein: the second op-amp provides the feedback signal to the second input port of the first op-amp and receives the amplified output at a first feedback input port; and the third op-amp provides a second feedback signal to the first input port of the first op-amp and to a second feedback input port of the second op-amp.

In some such embodiments, a second feedback signal of the common voltage is provided to second input port of the first op-amp and the feedback signal is provided to the second input port of the first op-amp.

In some such embodiments, the DC coupled analog front end further comprises a feedback resistor and a feedback capacitor arranged in series between the feedback stage and the second input port of the first op-amp.

In some such embodiments, the DC coupled analog front end further comprises a resistive feedback loop, connected between the output port of the first op-amp and the second input port of the first op-amp that provides the amplified output as at least a portion of the feedback signal.

In some such embodiments, the DC coupled analog front end further comprises a capacitive feedback loop, connected between the output port of the first op-amp and the second input port of the first op-amp that provides the amplified output as at least a portion of the feedback signal.

In some such embodiments, the DC source has a voltage of 2.5 Volts.

In one embodiment, a Direct Current (DC) coupled analog front end for an electrocardiogram (ECG) measuring device is provided, comprising: an ECG patch input; a first op-amp having a first input port, a second input port, and a first output port; a second op-amp having a third input port, a fourth input port, and a second output port; and a DC source, wherein: the DC source is coupled between the ECG patch input and the first input port; the ECG patch input is in communication with the first input port; the first output port is in communication with the fourth input port.

In some such embodiments, the DC coupled analog front end further comprises an ECG signal output; and a passive lowpass filter, wherein the passive lowpass filter in communication between the first output port and the signal output.

In some such embodiments, the DC coupled analog front end further comprises: an input resistor; and an input capacitor, wherein: the second op-amp is an inverting op-amp; the fourth input port is an inverting input port; the third port is in communication with a common voltage source; the input resistor is disposed between the first output port and the fourth input port; and the input capacitor is disposed between the third input port and the second output port.

In some such embodiments, the DC coupled analog front end further comprises: a third op-amp, having a fifth input port, a sixth input port, and a third output port; wherein: the second output port is in communication with the first input port; the third output port is in communication with the second input port; the fifth input port is in communication with the common voltage source; and the sixth input port is in communication with the second output port.

In some such embodiments, the DC coupled analog front end further comprises: a common voltage source; and a third op-amp having a fifth input port, a sixth input port, and a third output port; wherein: the second output port is in communication with the third input port and the third output port; the fifth input port is in communication with the common voltage source; the third output port is in communication with the first input port and the sixth input port.

In some such embodiments, the third output port is in communication with a negative terminal of the DC source; and the first output port is in communication with the second input port.

In some such embodiments, the DC coupled analog front end, further comprises: a feedback resistor and a feedback capacitor arranged in series between the second output port and the second input port.

In one embodiment, a wearable electrocardiography monitor, is provided comprising: an extended wear electrode patch including a battery, an electrocardiography (ECG) patch input, and a Direct Current (DC) analog front end, the DC analog front end including: a first op-amp having a first input port, a second input port, and a first output port; and a second op-amp having a third input port, a fourth input port, and a second output port; wherein: the battery is coupled between the ECG patch input and the first input port; the ECG patch input is in communication with the first input port; the first output port is in communication with the fourth input port; and the second output port is in communication with the ECG patch input.

In some such embodiments, the DC analog front end includes: a common voltage source; a third op-amp having a fifth input port, a sixth input port, and a third output port, wherein: the second output port is in communication with the first input port; the third output port is in communication with the second input port; the fifth input port is in communication with the common voltage source; and the sixth input port is in communication with the second output port.

In some such embodiments, the DC analog front end includes: a common voltage source; a third op-amp having a fifth input port, a sixth input port, and a third output port, wherein: the second output port is in communication with the third input port and the third output port; the fifth input port is in communication with the common voltage source; and the third output port is in communication with the first input port and the sixth input port.

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, including time and clustering of events, all without departing from their spirit and the scope. Accordingly, the drawings and detailed description are to be regarded as illustrative in nature and not as restrictive.

### BRIEF DESCRIPTION OF THE DRAWINGS

FIG. 1 illustrates an example of an electrocardiography monitor, including an extended wear electrode patch fitted to the sternal region of a human male biological subject, according to embodiments of the present disclosure.

FIG. 2 is an example circuit design for a first stage of an electrocardiogram (ECG) analog front end (AFE), according to embodiments of the present disclosure.

FIG. 3 illustrates an example feedback stage as may be used in conjunction with the example amplifying stage shown in FIG. 2, according to embodiments of the present disclosure.

FIG. 4 illustrates an example feedback stage as may be used in conjunction with the example amplifying stage shown in FIG. 2, according to embodiments of the present disclosure.

FIG. 5 illustrates an example feedback stage as may be used in conjunction with the example amplifying stage shown in FIG. 2, according to embodiments of the present disclosure.

FIG. 6 illustrates an example feedback stage as may be used in conjunction with the example amplifying stage shown in FIG. 2, according to embodiments of the present disclosure.

FIG. 7 illustrates digital signals used by the ECG monitor described herein for measuring an ECG waveform, according to embodiments of the present disclosure.

FIG. 8 illustrates an example computing device, according to embodiments of the present disclosure.

### DETAILED DESCRIPTION

The present disclosure provides enhanced electrocardiogram (ECG) analog front end (AFE). An ECG measurement patch adhered to the skin for the longitudinal analysis of a biological subject's ECG over an extended duration (e.g., a month or more) needs to be powered over the length of analysis period, which may require the replacement, recharging, or conservation of battery power in the device. As these devices are adhered to the skin of the biological subject, design goals frequently prioritize reducing power consumption, which reduces the size and weight of the needed battery and avoid the need to replace or recharge the battery, which in turn reduces the risk of an operator forgetting to replace/recharge the battery, improves adhesion to the biological subject's skin, reduces discomfort of wearing the devices, and various other benefits. However, conventional ECG AFEs often make sacrifices in one or more design goals, which may also include the responsiveness, accuracy, and cost of the devices used therein. Accordingly, conventional implementations offer suffer from one or more of high-power consumption, differential thermal noise, high noise (e.g., due to difference potential of the right-leg-drive (RLD) and the reference of the input stage, and complex designs with associated high costs and large sizes. Accordingly, the described devices are smaller and lighter and have a power source that can last over a longer duration that previous devices, among other benefits offered.

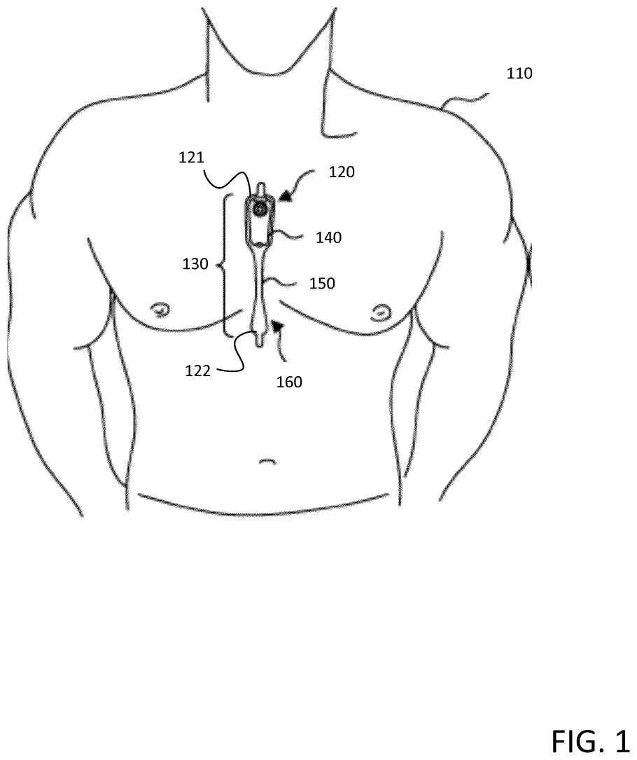

FIG. 1 illustrates an example of an electrocardiography monitor, including an extended wear electrode patch fitted to the sternal region of a human male biological subject, according to embodiments of the present disclosure. ECG and physiological monitoring can be provided through a wearable ambulatory monitor that includes two components, a flexible extended wear electrode patch and a removable reusable (or single use) monitor recorder. Both the electrode patch and the monitor recorder are configured to capture electrical signals from the propagation of low amplitude, relatively low frequency content cardiac action potentials, such as the P-waves generated during atrial activation. By way of example, FIG. 1 shows an extended wear electrocardiography monitor 120, including a monitor recorder 140. The wearable monitor 120 sits centrally, positioned axially along the sternal midline 160, on the subject's chest along the sternum 130 and oriented top-to-bottom with the monitor recorder 140 situated (preferably) towards the subject's head.

The electrode patch 150 is shaped to fit comfortably and conformal to the contours of the subject's chest approximately centered on the sternal midline 160 (or immediately to either side of the sternum 130). The distal end of the electrode patch 150, under which a lower or inferior pole 122 (ECG electrode) is adhered, extends towards the Xiphoid process and lower sternum, and (depending upon the subject's build) may straddle the region over the Xiphoid process and lower sternum. The proximal end of the electrode patch 150, located under the monitor recorder 140, under which an upper or superior pole 121 (ECG electrode) is adhered, is below the manubrium and, depending upon subject's build, may straddle the region over the manubrium.

Although discussed herein primarily with reference to a biological subject that is human, the present disclosure contemplates that the teachings provided herein may be applied for electrocardiographic devices used with non-human biological subjects. Accordingly, the various anatomical terms used to describe physiological features and relative locations in a human model may be modified to describe recognizably similar physiological features and relative locations in non-human models.

During ECG monitoring, the amplitude and strength of action potentials sensed on the body's surface are affected to varying degrees by cardiac, cellular, extracellular, vector of current flow, and physical factors, like obesity, dermatitis, high impedance skin, etc. Sensing along the sternal midline 160 (or immediately to either side of the sternum 130) significantly improves the ability of the wearable monitor 120 to cutaneously sense cardiac electric signals, particularly the P-wave (or atrial activity) and, to a lesser extent, the QRS interval signals in the ECG waveforms that indicate ventricular activity by countering some of the effects of these factors. Depending upon their placement locations on the chest, ECG electrodes may be separated from activation regions within the heart by differing combinations of internal tissues and body structures, including heart muscle, intracardiac blood, the pericardium, intrathoracic blood and fluids, the lungs, skeletal muscle, bone structure, subcutaneous fat, and the skin, plus any contaminants present between the skin's surface and electrode signal pickups. The degree of amplitude degradation of cardiac transmembrane potentials increases with the number of tissue boundaries between the heart and the skin's surface that are encountered. The cardiac electrical field is degraded each time the transmembrane potentials encounter a physical boundary separating adjoining tissues due to differences in the respective tissues' electrical resistances. In addition, other non-spatial factors, such as pericardial effusion, emphysema or fluid accumulation in the lungs, can further degrade body surface potentials.

These degradations in the heartbeat signal due to effects from the physiology of the biological subject all decrease the Signal to Noise Ratio (SNR) in generating an ECG; either by increasing noise magnitude or decreasing signal magnitude.

The wearable monitor 120 may be understood as having two patch connections that operate together to provide the functionalities described herein. The first patch connection is primarily the ECG analog front end, which receives an input from an electrode near or connected to the inferior pole 122. The input is connected to an electrode near the inferior pole 122. The output of the first patch is filtered to produce a low frequency rejected ECG signal that goes to an Analog-to-Digital Converter in the Micro Control Unit (MCU).

The second patch connection describes the driving circuitry that drives the second electrode that is near or connected to the superior pole 121. This driving circuitry drives the second electrode at the midpoint of the analog front end supply, and ultimately serves as a reference signal for the first patch connection (e.g., signal output 220).

Both patches may use the same battery and ground, and may be constructed on the same printed circuit board as one another. As part of self-testing, the MCU drives the second patch with a predefined voltage/current pattern and that drives a signal pass through the body of a biological subject wearing the wearable monitor 120, which is sensed as part of the ECG signal through the first patch.

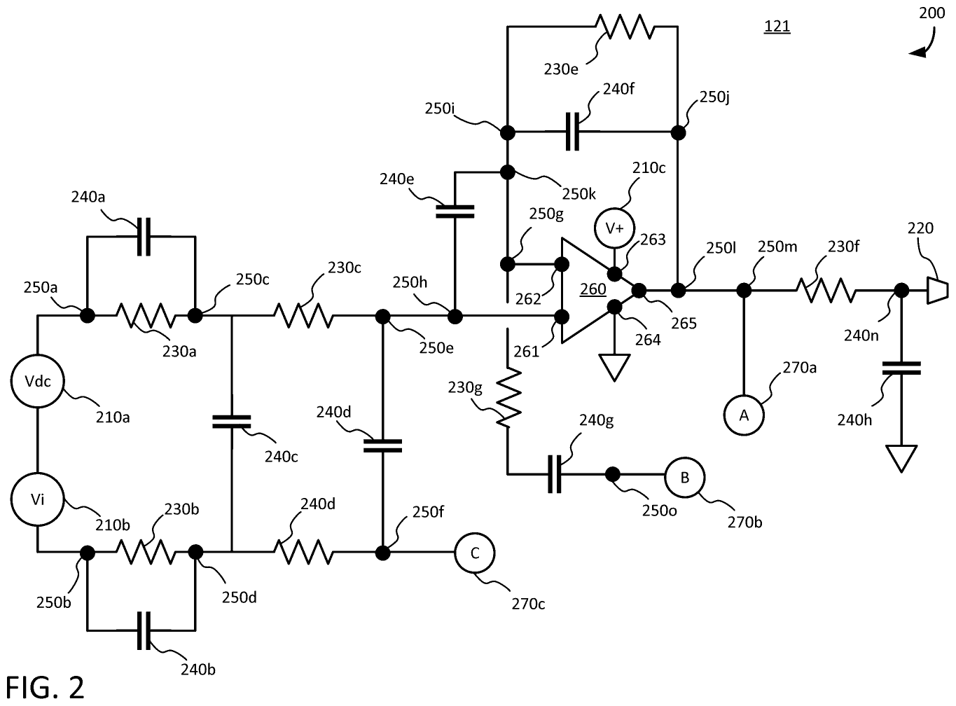

FIG. 2 is a circuit diagram example of an amplifying stage 200 that is located in, or in electrical communication with, a superior pole 121 of a wearable monitor 120, such as the example shown in FIG. 1, that is positioned superior to the xiphoid process of the subject 110 and inferior to or proximal to the manubrium of the subject 110. In some embodiments, some or all of the electrical components of the amplifying stage 200 are located in a monitor recorder 140 of the wearable monitor 120.

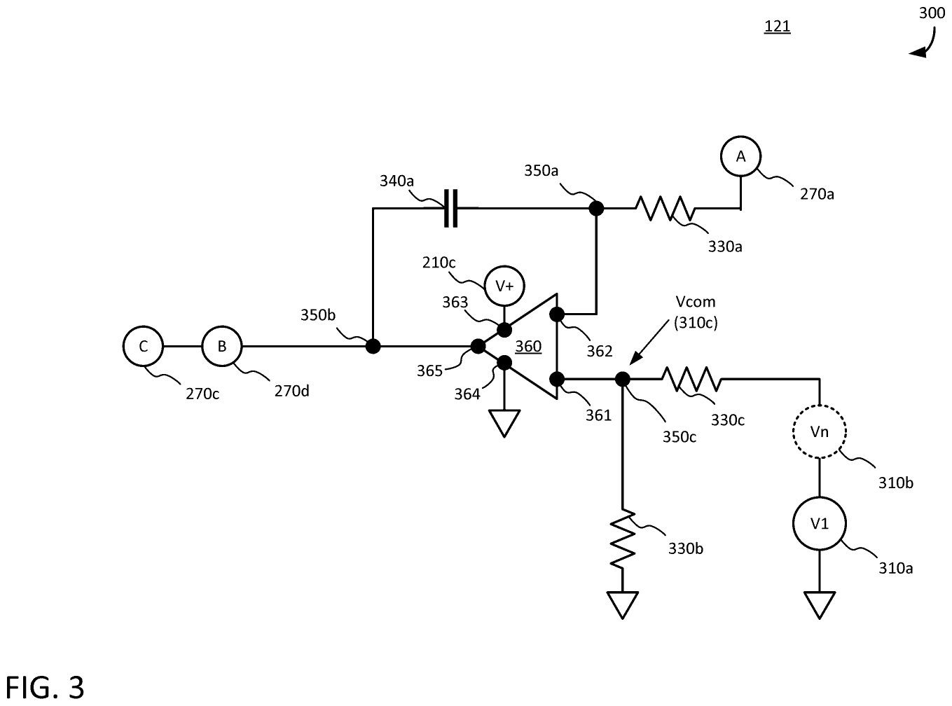

In FIGS. 2-6, various components are designated by like numerals and unique letter designators to distinguish, the various components from one another, but not all components may be included in every embodiment, and some components may be presented to allow for simulation of the circuit (e.g., to introduce noise, to simulate resistances or capacitances in other elements), but are omitted in the physical apparatus thereof. For example, FIG. 2 may refer specifically to a first resistor 230a or a second resistor 230b, or generally refer to resistors 230. Similarly, FIG. 3 may refer to a first capacitor 340a and a second capacitor 340b (regardless of whether two capacitors 340 are included in the illustrated circuit diagram), or generally refer to capacitors 340.

As illustrated in FIG. 2, the amplifying op-amp 260 shown with a correspondingly designated set of ports, with a first input port 261, a second input port 262, a positive supply port 263, a negative supply port 264, and an output port 265. As illustrated, the negative supply port 264 is grounded, and the positive supply port 263 is connected to a third voltage input 210c of a driving voltage (V+), which in various embodiments, supplies a 2.5 Volt (V) Direct Current (DC) power.

A coupled DC voltage source 210a (e.g., from a battery power source for the patch) and an input patch voltage source 210b (e.g., measuring the ECG signal of a wearer of the patch) are provided in series between the first node 250a (e.g., a positive node) and a second node 250b (e.g., a negative node). In various embodiments, a first resistor 230a and a first capacitor 240a are included in the design to represent the resistance and capacitance of the skin of the patient from whom the ECG signal is being measured, and may be included in parallel between the first node 250a and a third node 250c for purposes of simulation. Similarly, in various embodiments, a second resistor 230b and a second capacitor 240b are included in the design to represent the reference resistance and capacitance of the patch, and may be included in parallel between the second node 250b and a fourth node 250d.

The third resistor 230c, the fourth capacitor 240d, and the skin-electrode impedance (e.g., of approximately 51 kΩ), form a 9 kHz LPF, while the fifth capacitor 240e provides a shunting path for electromagnetic interference.

The fifth resistor 230e and the sixth capacitor 240f add a LPF pole at around 45 Hz in some embodiments, although the pole may be shifted by changing the resistance and capacitance placed between the ninth node 250i and the tenth node 250j.

The amplifying op-amp 260 and the feedback network formed by the fifth resistor 230c, sixth capacitor 240f, seventh resistor 230g, and the seventh capacitor 240g serves as the amplifying stage 200. In various embodiments, this amplifying stage 200 provides the new AFE and has a gain of 330 using a reference voltage of 2.5 V. The fifth resistor 230e and sixth capacitor 240f block the DC path of the amplify stage 200, adds a HPF pole and a zero to the system.

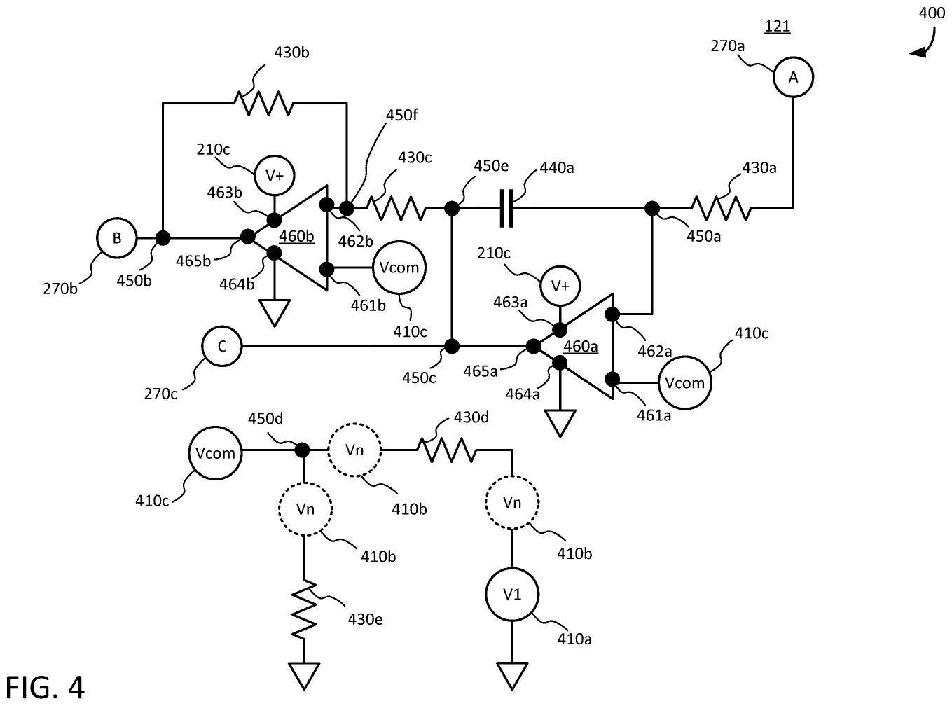

The input resistor (e.g., resistor 330a in FIG. 3, 430a in FIG. 4, etc.) and input capacitor (e.g., capacitor 340a in FIG. 3, 440a in FIG. 4, etc.) add a High Pass Filter (HPF) pole to feedback stage the system. The poles and zeros added by the fifth resistor 230e and sixth capacitor 240f from the amplifying stage 200 together with the feedback stage input resistor and capacitor determine the systems DC rejection corner frequency, and low frequency roll up region behavior. The high frequency rejection is determined by the poles added by the third resistor 230c, fifth resistor 230c, sixth capacitor 240f, and fourth capacitor 240d. Ideally the integrator should be configured to have limited gain at DC, to mitigate the finite gain of a real op-amp. Because the integrator output is negatively fed back to the input signal and eventually has a limited close loop gain and the system is receiving dynamic signals, the gain limiting is not necessary. In addition, large gain requires a smaller feedback resistor, and because the voltage across that resistor can be as large as 300 mV, the additional power consumption is unacceptable.

One variation of the design is to change from resistive feedback to capacitive feedback. Because of the higher variation in manufacturing tolerances of capacitors compared to resistors, this variation is a tradeoff between better passband flatness (capacitive feedback) and better passband corner frequency accuracy (resistive feedback). Because of the better granularity of resistors available, resistor variation offers better corner frequency tuning granularity. Capacitive feedback consumes slightly higher dynamic power. If thick film resistors can be used based on noise measurement, the dynamic power can be reduced.

The ECG benchmark has shown that the passband of the system is not sensitive to small passband corner frequency shifts, therefore resistive feedback is chosen. The design can use the same footprint components to switch between these two variations should one show clear advantages over the other.

An additional passive anti-aliasing (lowpass) filter can optionally be added (at the trade-off of extra power consumption) to the output of the amplifying op-amp 260, which include the sixth resistor 230f and eighth capacitor 240h, between which the eighth node 250h is disposed, from which the signal output 220 of the front end may be taken. This passive single pole anti-aliasing filter may improve compression ratio at a cost of added power consumption.

In both resistive feedback and capacitive feedback variations, voltage across the seventh resistor 230g and seventh capacitor 240g has a small amount of AC energy centered around Vcom; therefore, the dynamic power consumption is negligible. For the path, over the fifth resistor 230e and the sixth capacitor 240f, if the sixth capacitor 240f has a capacitance around 10 nF, impedance is around 31.8 kΩ at 50 Hz, and 318 kΩ at 5 Hz respectively. The equivalent rms impedance of the path can be estimated by

$${Z}_{c 2 rms} = ( 31.8 k ‖ 10 M ) \times \frac{\sqrt{2}}{2} \times 10 \% + ( 318 k ‖ 10 M ) \times \frac{\sqrt{2}}{2} \times 90 \% = 198 k Ω .$$

If the sixth capacitor 240f has a capacitance around 3.3 nF, impedance is around 964 kΩ at 50 Hz and 9.64 MΩ at 5 Hz, and the rms impedance of the path can

$${Z}_{c 2 rms} = ( 964 k ‖ 1 M ) \times \frac{\sqrt{2}}{2} \times 10 \% + ( 9.64 M ‖ 1 M ) \times \frac{\sqrt{2}}{2} \times 90 \% = 611 k Ω .$$

### That is

$$\frac{1.26 \times \frac{\sqrt{2}}{2}}{198 k} = 4.5 μA and \frac{126 \times \frac{\sqrt{2}}{2}}{611 k} = 1 \text{.45} μA ,$$

respectively.

The desired overall system lowpass cutoff is at 45 Hz which is dominated by f_{H}_{_{A}}_{LPF}. To reduce the sensitivity of parasititic capacitance, the sixth capacitor 240f has experimentally been modeled with a capacitance of 3.3 nF, and the fifth resistor 230e has been modeled with a resistance of 1 MΩ. A higher cut in frequency adds risk of not being able to recover low frequency content. Therefore, the desired overall system cut in frequency should be below 2.4 Hz.

In order to maintain a 20 dB/decade roll off near DC (DC rejection), f_{H}_{_{β}}_{0dB}>f_{H}_{_{A}}_{0dB}. If f_{H}_{_{β}}_{0dB}<f_{H}_{_{A}}_{0dB}, the weak DC rejection may result in baseline wander. There is no benefit to move f_{H}_{_{β}}_{HPF} close to f_{H}_{_{A}}_{HPF}, therefore, overall system's cut in frequency should be determined by f_{H}_{_{A}}_{HPF}.

An option is to use 10 MΩ thick film resistor. Since the dynamic voltage across the feedback input resistor is not negligible, excess noise due to voltage stress on thick film resistor may be unacceptable. If noise is acceptable, higher resistance with smaller capacitors offer less variation of component. Production calibration in this case is more beneficial because both the capacitances of the seventh capacitor 240g and the feedback stage input capacitor can be determined and compensated for.

Unlike previous AFEs (which use a reference voltage of 2.3 V), the present design uses a reference voltage of 2.5 V. The quantization noise difference between 2.3V and 2.5V is

$$Δ {V}_{noiserms} = \frac{2.5 - 2.3}{{2}^{n}} \times \frac{1}{\sqrt{12}} .$$

For 14 ENOB ADC, ΔVₙₒᵢₛₑᵣₘₛ≈3 μVᵣₘₛ. That is less than 1 nVᵣₘₛ input referred noise; therefore, the increased reference voltage does not affect system noise, while the higher dynamic range offers better signal resolution. Typical 3.3 V Flash does not allow write access below 2.7 V, considering a small dropout to maintain PSRR and parts availability, 2.5 V is the better option.

Note that in the HPF cutoff region, the input dynamic range is more than 7 mVpp. Because the new AFE's characteristics is less sensitive to variations (component variations, skin-electrode impedance), reconstructing of the contents in the HPF cutoff region is more accurate.

As a non-limiting example, each of the components in the example amplifying stage 200 may have about the following values.

TABLE 1
Component Reference Value

| Component | Value | |
|---|---|---|
| Voltage Source (V_{DC}) 210a | 2.5 | V |
| Voltage Source (Vᵢ) 210b | Variable | |
| Voltage Source (V+) 210c | 2.5 | V |
| Resistor 230a | Variable Ω | |
| Resistor 230b | Variable Ω | |
| Resistor 230c | 68.1 | kΩ |
| Resistor 230d | 68.1 | kΩ |
| Resistor 230e | 1 | MΩ |
| Resistor 230f | 330 | kΩ |
| Resistor 230g | 107 | Ω |
| Capacitor 240a | Variable μF | |
| Capacitor 240b | Variable μF | |
| Capacitor 240c | 10 | pF |
| Capacitor 240d | 100 | pF |
| Capacitor 240e | 10 | pF |
| Capacitor 240f | 100 | nF |
| Capacitor 240g | 33 | μF |
| Capacitor 240h | 0.01 | μF |

As a non-limiting example, each of the components in the example amplifying stage 200 may have about the following values.

TABLE 2
Component Reference Value

| Component | Value | |
|---|---|---|
| Voltage Source (V_{DC}) 210a | 2.5 | V |
| Voltage Source (Vᵢ) 210b | Variable | |
| Voltage Source (V+) 210c | 2.5 | V |
| Resistor 230a | Variable Ω | |
| Resistor 230b | Variable Ω | |
| Resistor 230c | 68.1 | kΩ |
| Resistor 230d | 0.001 | Ω |
| Resistor 230e | 1 | MΩ |
| Resistor 230f | 330 | kΩ |
| Resistor 230g | 3 | kΩ |
| Capacitor 240a | Variable μF | |
| Capacitor 240b | Variable μF | |
| Capacitor 240c | 10 | pF |
| Capacitor 240d | 100 | pF |
| Capacitor 240e | 10 | pF |
| Capacitor 240f | 0.0033 | μF |
| Capacitor 240g | 47 | μF |
| Capacitor 240h | 0.01 | μF |

FIG. 3 illustrates an example feedback stage 300 as may be used in conjunction with the example amplifying stage 200 shown in FIG. 2, according to embodiments of the present disclosure. A feedback op-amp 360 may be configured as an inverter with unity gain, that is applied via the second interface 270b and the third interface 270c back to the amplifying stage 200. A bypass line is defined between the first node 350a and the second node 350b, which includes the first capacitor 340a. Although illustrated with a capacitive bypass line, in various embodiments, the bypass line may be resistive (e.g., omitting the first capacitor 340a for another resistor 330) or resistive and capacitive (e.g., including another resistor 330 in series with the first capacitor 340a between the first node 350a and the second node 350b).

A first resistor 330a is disposed between the first interface 270a and the first node 350a, which is connected to the second port 362 of the feedback op-amp 360 (e.g., an inverting input port). A common voltage (Vcom) source 310c is applied to the first port 361 of the feedback op-amp 360. In various embodiments, the common voltage is generated at the third node 350c, which is connected to ground via a second resistor 330b, and is separated via a third resistor 330c from a first voltage source 310a that produces a DC voltage of V1 via a third resistor 330c. In various embodiments, the third resistor 330c and the second resistor 330b have substantially equal resistances, thereby providing Vcom as half of V1. For example, when V1 is 2.5 Volts DC (V_{DC}), Vcom may be 1.25 V_{DC}.

In various embodiments, a second voltage source 310b may optionally be included when evaluating the design under simulation to provide a noise voltage (Vn) at various points on the feedback stage 300 to demonstrate the robustness of the design to noise. As will be appreciated, various types of signal noise may be imparted on the design, including white noise, flickering noise (with constant, variable, or cyclical frequencies), voltage floor offsets (with constant positive or negative amplitude differences from a nominal value), etc.

As a non-limiting example, each of the components in the example feedback stage 300 may have about the following values.

TABLE 3
Component Reference Value

| Component | Value | |
|---|---|---|
| Voltage Source (V1) 310a | 2.5 | V |
| Voltage Source (Vn) 310b | Variable | |
| Resistor 330a | 1 | MΩ |
| Resistor 330a | 1 | MΩ |
| Resistor 330a | 1 | MΩ |
| Capacitor 340a | 47 | μF |

FIG. 4 an example feedback stage 400 as may be used in conjunction with the example amplifying stage 200 shown in FIG. 2, according to embodiments of the present disclosure. The example feedback stage 400 shown in FIG. 4 expands on the example feedback stage 300 shown in FIG. 3 by adding a second feedback op-amp 460b between the output of the feedback op-amp 360 (e.g., the first feedback op-amp 460a) and the second interface 270b.

Another variation is to add an inverting amplifier to further improve transient response. This variant would have comparable power consumption and noise level as the current front ends, but with much better transient response.

The first feedback op-amp 460a may be configured as an inverter with unity gain, that is applied via the third interface 270c back to the amplifying stage 200. A first resistor 430a is disposed between the first interface 270a and the first node 450a, which is connected to the second port 462 of the first feedback op-amp 460a (e.g., an inverting input port). A bypass line is defined between the first node 450a and the third node 450c, which includes the first capacitor 440a. Although illustrated with a capacitive bypass line, in various embodiments, the bypass line may be resistive (e.g., omitting the first capacitor 440a for another resistor 430) or resistive and capacitive (e.g., including another resistor 430 in series with the first capacitor 440a between the first node 450a and the third node 450c).

A common voltage (Vcom) source 410c is applied to the first port 461a of the first feedback op-amp 460. In various embodiments, the common voltage is generated at the fourth node 450d, which is connected to ground via a fifth resistor 430e, and is separated via a fourth resistor 430d from a first voltage source 410a that produces a DC voltage of V1. In various embodiments, the fourth resistor 430d and the fifth resistor 430e have substantially equal resistances, thereby providing Vcom as half of V1. For example, when V1 is 2.5 V_{DC}, Vcom may be 1.25 V_{DC}.

The second op-amp 460b may also be configured as an inverter with unity gain, which receives input of the output of the first feedback op-amp 460a via a third resistor 430c to the second port 462b. This third resistor 430c may be understood as being disposed between a fifth node 450c (shared with the third node 450c) and a sixth node 450f that is connected to the second port 462b.

The common voltage Vcom is applied to the first port 461b of the second op-amp 460b. The output from the second op-amp 460b is delivered to the second node 450b and the second interface 270b back to the amplifying stage 200. A bypass line is defined between the sixth node 450f and the second node 450b, which includes the second resistor 430b. Although illustrated with a resistive bypass line, in various embodiments, the bypass line may be capacitive (e.g., omitting the second resistor 430b for another capacitor 440) or resistive and capacitive (e.g., including another capacitor 440 in series with the second resistor 430b between the sixth node 450f and the second node 450b).

In various embodiments, a second voltage source 410b may optionally be included when evaluating the design under simulation to provide a noise voltage (Vn) at various points on the feedback stage 400 to demonstrate the robustness of the design to noise. As will be appreciated, various types of signal noise may be imparted on the design, including white noise, flickering noise (with constant, variable, or cyclical frequencies), voltage floor offsets (with constant positive or negative amplitude differences from a nominal value), etc.

As a non-limiting example, each of the components in the example feedback stage 400 may have about the following values.

TABLE 4
Component Reference Value

| Component | Value | |
|---|---|---|
| Voltage Source (V1) 410a | 2.3 | V |
| Voltage Source (Vn) 410b | Variable | |
| Resistor 430a | 1 | MΩ |
| Resistor 430b | 10 | kΩ |
| Resistor 430c | 10 | kΩ |
| Resistor 430d | 1 | MΩ |
| Resistor 430e | 1 | MΩ |
| Capacitor 440a | 47 | μF |

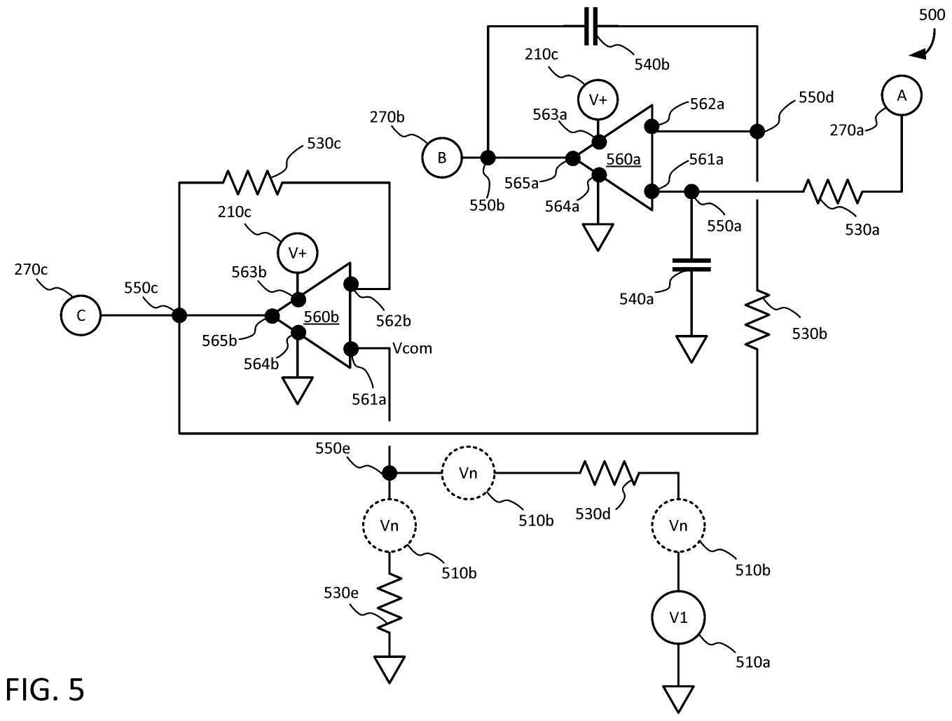

FIG. 5 illustrates an example feedback stage 500 as may be used in conjunction with the example amplifying stage 200 shown in FIG. 2, according to embodiments of the present disclosure. A first feedback op-amp 560a may be configured with unity gain, that is applied via the second interface 270b back to the amplifying stage 200. An input line feeds the output from the amplifying stage 200 via the first interface 270a to the first port 561a of the first feedback op-amp 560a via a first resistor 530a to a first node 550a. The first node 550a may be connected to ground via a first capacitor 540a.

Another variant is to reject DC voltage at the amplifying stage, without going through the patient, which eliminates the effect of impedance characteristic of the patient. This design has similar level of power consumption and noise level as the current front ends.

The second port 562a of the first feedback op-amp 560a may be an inverting port, and is fed from a fourth node 550d. A bypass line for the first feedback op-amp 560a is defined between the fourth node 550d and the second node 550b, which includes the second capacitor 540b. Although illustrated with a capacitive bypass line, in various embodiments, the bypass line may be resistive (e.g., omitting the second capacitor 540b for another resistor 330) or resistive and capacitive (e.g., including another resistor 530 in series with the second capacitor 540b between the fourth node 550d and the second node 550b).

The output of a second feedback op-amp 560b is fed, via a third node 550c, to the amplifying stage 200 via the third interface 270c and to the first feedback op-amp 560a at the fourth node 550d, which is separated from the third node 550c by a second resistor 530b. The second feed-back op-amp 560b may be configured as an inverting/integrating op-amp with unity gain, which receives a common voltage Vcom at a first port 562a and the output of the third interface 270c and the output of the second feedback op-amp 560c at a the third node 550c via a third resistor 530c disposed between the second port 562b and the third node 550c.

In various embodiments, the common voltage is generated at the fifth node 550e, which is connected to ground via a fifth resistor 530e, and is separated via a fourth resistor 530d from a first voltage source 510a that produces a DC voltage of V1. In various embodiments, the fourth resistor 530d and the fifth resistor 530e have substantially equal resistances, thereby providing Vcom as half of V1. For example, when V1 is 2.5 Volts DC (V_{DC}), Vcom may be 1.25 V_{DC}.

In various embodiments, a second voltage source 510b may optionally be included when evaluating the design under simulation to provide a noise voltage (Vn) at various points on the feedback stage 500 to demonstrate the robustness of the design to noise. As will be appreciated, various types of signal noise may be imparted on the design, including white noise, flickering noise (with constant, variable, or cyclical frequencies), voltage floor offsets (with constant positive or negative amplitude differences from a nominal value), etc.

As a non-limiting example, each of the components in the example feedback stage 500 may have about the following values.

TABLE 5
Component Reference Value

| Component | Value | |
|---|---|---|
| Voltage Source (V1) 510a | 1.2 | V |
| Voltage Source (Vn) 510b | Variable | |
| Resistor 530a | 1 | MΩ |
| Resistor 530b | 1 | MΩ |
| Resistor 530c | 100 | Ω |
| Resistor 530d | 1 | MΩ |
| Resistor 530e | 1 | MΩ |
| Capacitor 540a | 47 | μF |
| Capacitor 540b | 2.2 | μF |

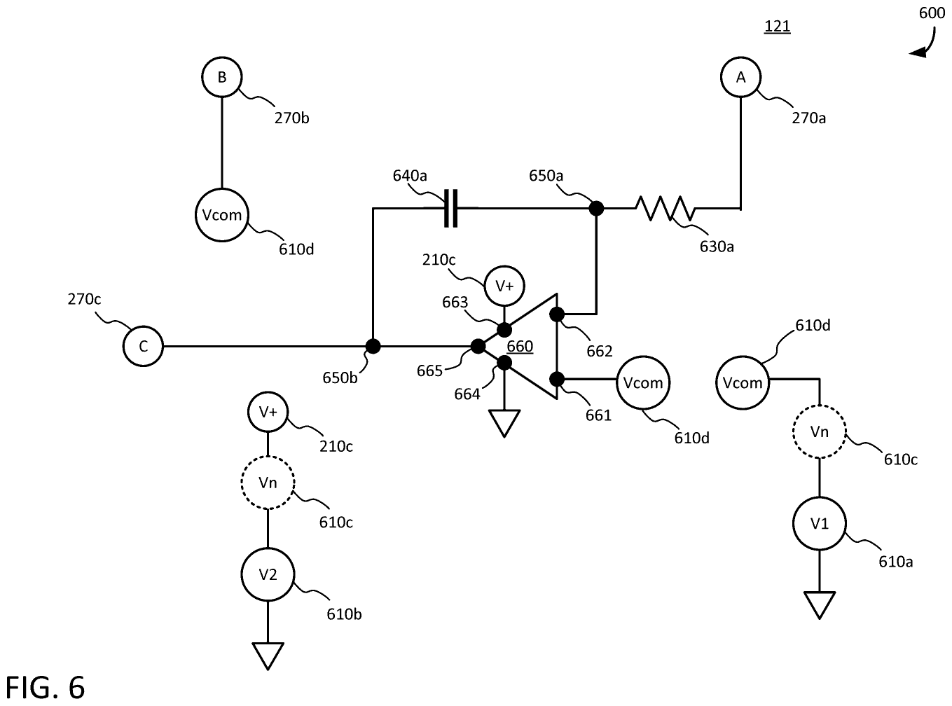

FIG. 6 illustrates an example feedback stage 600 as may be used in conjunction with the example amplifying stage 200 shown in FIG. 2, according to embodiments of the present disclosure. A feedback op-amp 660 may be configured with inverting unity gain, that is applied via the third interface 270c back to the amplifying stage 200. A first resistor 630a is disposed between the first interface 270a and the first node 650a, which is connected to the second port 662 of the feedback op-amp 660 (e.g., an inverting input port). A bypass line is defined between the first node 650a and the second node 650b, which includes the first capacitor 640a. Although illustrated with a capacitive bypass line, in various embodiments, the bypass line may be resistive (e.g., omitting the first capacitor 640a for another resistor 630) or resistive and capacitive (e.g., including another resistor 630 in series with the first capacitor 640a between the first node 650a and the second node 650b).

Another variation is add an LDO that outputs at VCOM, which is the midpoint of the ADC supply. This design variant adds a couple hundred nA due to LDO quiescent current. This variant offers significant improvement on transient response, at potential cost of worse Power Supply Rejection performance because the power supply noise is only rejected by op-amp, compared to the other variants where the power supply noise is rejected by the LDO first and then by op-amp. Considering that the system is extremely low power, it is possible that the PSRR is still acceptable.

A common voltage (Vcom) source 610d is applied to the first port 641 of the feedback op-amp 660 and fed back as a reference voltage to the amplifying stage 200 via the second interface 270b. In various embodiments, the common voltage is generated by a first voltage source 610a that produces a DC voltage of V1, which may be affected (in practice or in simulation) by a noise voltage Vn, generated by a third voltage source 610c. As will be appreciated, various types of signal noise may be imparted on the design, including white noise, flickering noise (with constant, variable, or cyclical frequencies), voltage floor offsets (with constant positive or negative amplitude differences from a nominal value), etc.

A second voltage source 610b (which may be affected by the same or different voltage noise via a third voltage source 610c as the first voltage source 610a). The second voltage source 610b may provide the V+ voltage source 210b used to drive the feedback op-amp 660 and the op-amp 260 of the amplifying stage 200. In various embodiments, the voltage of the second voltage source 610b is double that of the first voltage source 610a, and the two voltage sources 610a-b may instead be provided via a single voltage source and voltage splitter.

As a non-limiting example, each of the components in the example feedback stage 600 may have about the following values.

TABLE 6
Component Reference Value

| Component | Value |
|---|---|
| Voltage Source (V1) 610a | 1.2 V |
| Voltage Source (V2) 610b | 2.4 V |
| Voltage Source (Vn) 610c | Variable |
| Resistor 630a | Variable |
| Capacitor 640a | Variable |

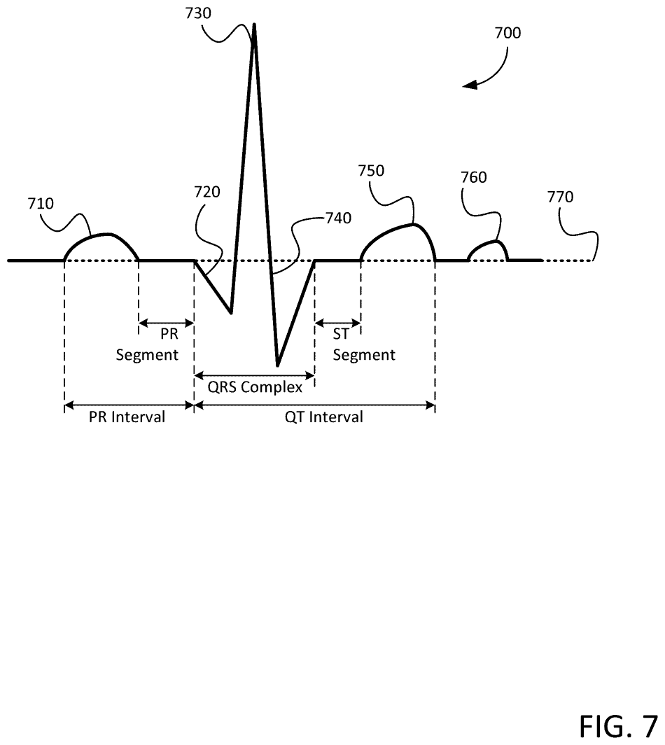

FIG. 7 illustrates components of an example ECG waveform 700, on which the x-axis represents time, and the y-axis represents cutaneous electrical signal strength relative to a baseline 770. The P-wave 710 has a smooth, normally upward, that is, positive, waveform that indicates atrial depolarization. The QRS complex often begins with the downward deflection of a Q-wave 720, followed by a larger upward deflection of an R-wave 730, and terminated with a downward waveform of the S-wave 740, collectively representative of ventricular depolarization. The T-wave 750 is normally a modest upward waveform, representative of ventricular depolarization, while the U-wave 760, often not directly observable, indicates the recovery period of the Purkinje conduction fibers.

Sampling of the intervals between successive waveforms 700 enables heart rate information derivation. For instance, the R-to-R interval represents the ventricular rate and rhythm, while the P-to-P interval represents the atrial rate and rhythm. Importantly, the PR interval is indicative of atrioventricular (AV) conduction time and abnormalities in the PR interval can reveal underlying heart disorders, thus representing another reason why the P-wave quality achievable by the ambulatory electrocardiography monitoring patch optimized for capturing low amplitude cardiac action potential propagation described herein is medically unique and important. The long-term observation of these ECG indicia, as provided through extended wear of the wearable monitor 120, provides valuable insights to the subject's cardiac function symptoms, and overall well-being, which may be used for the treatment or prophylaxis of various conditions.

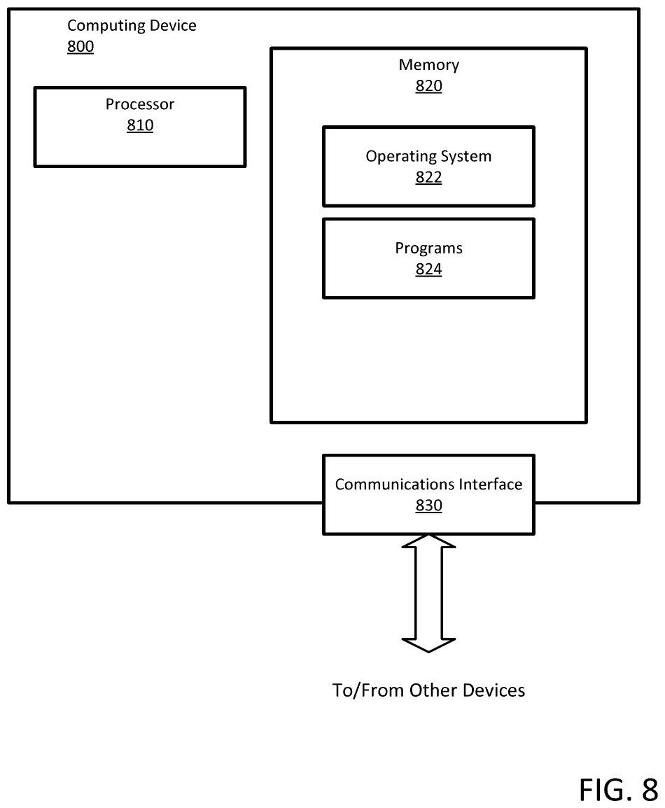

FIG. 8 illustrates a computing device 800, as may be used as an MCU to collect, collate, and output digital measurements of an ECG signal to produce an ECG, according to embodiments of the present disclosure. The computing device 800 may include at least one processor 810, a memory 820, and a communication interface 830.

The processor 810 may be any processing unit capable of performing the operations and procedures described in the present disclosure. In various embodiments, the processor 810 can represent a single processor, multiple processors, a processor with multiple cores, and combinations thereof.

The memory 820 is an apparatus that may be either volatile or non-volatile memory and may include RAM, flash, cache, disk drives, and other computer readable memory storage devices. Although shown as a single entity, the memory 820 may be divided into different memory storage elements such as RAM and one or more hard disk drives. As used herein, the memory 820 is an example of a device that includes computer-readable storage media, and is not to be interpreted as transmission media or signals per se.

As shown, the memory 820 includes various instructions that are executable by the processor 810 to provide an operating system 822 to manage various features of the computing device 800 and one or more programs 824 to provide various functionalities to users of the computing device 800, which include one or more of the features and functionalities described in the present disclosure. One of ordinary skill in the relevant art will recognize that different approaches can be taken in selecting or designing a program 824 to perform the operations described herein, including choice of programming language, the operating system 822 used by the computing device 800, and the architecture of the processor 810 and memory 820. Accordingly, the person of ordinary skill in the relevant art will be able to select or design an appropriate program 824 based on the details provided in the present disclosure.

The communication interface 830 facilitates communications between the computing device 800 and other devices, which may also be computing devices as described in relation to FIG. 8. In various embodiments, the communication interface 830 includes antennas for wireless communications and various wired communication ports. The computing device 800 may also include or be in communication, via the communication interface 830, one or more input devices (e.g., a keyboard, mouse, pen, touch input device, etc.) and one or more output devices (e.g., a display, speakers, a printer, etc.).

Although not explicitly shown in FIG. 8, it should be recognized that the computing device 800 may be connected to one or more public and/or private networks via appropriate network connections via the communication interface 830. It will also be recognized that software instructions may also be loaded into a non-transitory computer readable medium such as the memory 820 from an appropriate storage medium or via wired or wireless means.

Accordingly, the computing device 800 is an example of a system that includes a processor 810 and a memory 820 that includes instructions that (when executed by the processor 810) perform various embodiments of the present disclosure. Similarly, the memory 820 is an apparatus that includes instructions that when executed by a processor 810 perform various embodiments of the present disclosure.

The present disclosure may also be understood with reference to the following numbered clauses.

Clause 1. A Direct Current (DC) coupled analog front end for an electrocardiogram (ECG) measuring device, comprising: an amplifying stage including a first op-amp that is configured to receive a signal input from an ECG patch at a first input port and a feedback signal at a second input port and to produce an amplified output of an ECG signal via a first output port; a feedback stage including a second op-amp that is configured to receive the amplified output and produce the feedback signal at a second output port; and a DC source, coupled between the signal input from the ECG patch and the first input port.

Clause 2. The DC coupled analog front end of clause 1, further comprising a passive lowpass filter in communication between the first output port of the first op-amp and a signal output for an ECG signal.

Clause 3. The DC coupled analog front end of clause 1, wherein the second op-amp is an inverting op-amp that receives the amplified output at an inverting input port and a common voltage at a feedback input port to produce the feedback signal, wherein an input resistor is disposed between the inverting input port and the first output port of the first op-amp and an input capacitor is disposed between the inverting input port and the second output port.

Clause 4. The DC coupled analog front end of clause 3, further comprising: a third op-amp disposed in the feedback stage, and configured to produce a second feedback signal that the first op-amp is configured to receive at the second input port with the feedback signal produced by the second op-amp received at the first input port of the first op-amp, the third op-amp receiving the feedback signal at a second inverting import port and the common voltage at a second feedback input port to produce the second feedback signal.

Clause 5. The DC coupled analog front end of clause 1, further comprising a third op-amp disposed in the feedback stage, wherein: the second op-amp provides the feedback signal to the second input port of the first op-amp and receives the amplified output at a first feedback input port; and the third op-amp provides a second feedback signal to the first input port of the first op-amp and to a second feedback input port of the third op-amp.

Clause 6. The DC coupled analog front end of clause 5, further comprising a common voltage source wherein a second feedback signal is provided to the common voltage source and the feedback signal is provided to the second input port of the first op-amp.

Clause 7. The DC coupled analog front end of clause 1, further comprising: a feedback resistor and a feedback capacitor arranged in series between the feedback stage and the second input port of the first op-amp.

Clause 8. The DC coupled analog front end of clause 1, further comprising: a resistive feedback loop, connected between the first output port of the first op-amp and the second input port of the first op-amp that provides the amplified output as at least a portion of the feedback signal.

Clause 9. The DC coupled analog front end of clause 1, further comprising: a capacitive feedback loop, connected between the first output port of the first op-amp and the second input port of the first op-amp that provides the amplified output as at least a portion of the feedback signal.

Clause 10. The DC coupled analog front end of clause 1, wherein the DC source has a voltage of 2.5 Volts.

Clause 11. A Direct Current (DC) coupled analog front end for an electrocardiogram (ECG) measuring device, comprising: an ECG patch input; a first op-amp having a first input port, a second input port, and a first output port; a second op-amp having a third input port, a fourth input port, and a second output port; and a DC source, wherein: the DC source is coupled between the ECG patch input and the first input port; the ECG patch input is in communication with the first input port; the first output port is in communication with the fourth input port.

Clause 12. The DC coupled analog front end of clause 11, further comprising: an ECG signal output; and a passive lowpass filter, wherein the passive lowpass filter in communication between the first output port and the signal output.

Clause 13. The DC coupled analog front end of clause 11, further comprising: an input resistor; and an input capacitor, wherein: the second op-amp is an inverting op-amp; the fourth input port is an inverting input port; the third port is in communication with a common voltage source; the input resistor is disposed between the first output port and the fourth input port; and the input capacitor is disposed between the third input port and the second output port.

Clause 14. The DC coupled analog front end of clause 13, further comprising: a third op-amp, having a fifth input port, a sixth input port, and a third output port; wherein: the second output port is in communication with the first input port; the third output port is in communication with the second input port; the fifth input port is in communication with the common voltage source; and the sixth input port is in communication with the second output port.

Clause 15. The DC coupled analog front end of clause 11, further comprising: a common voltage source; and a third op-amp having a fifth input port, a sixth input port, and a third output port; wherein: the second output port is in communication with the third input port and the third output port; the fifth input port is in communication with the common voltage source; the third output port is in communication with the first input port and the sixth input port.

Clause 16. The DC coupled analog front end of clause 15, wherein: the third output port is in communication with a negative terminal of the DC source; and the first output port is in communication with the second input port.

Clause 17. The DC coupled analog front end of clause 1, further comprising: a feedback resistor and a feedback capacitor arranged in series between the second output port and the second input port.

Clause 18. A wearable electrocardiography monitor, comprising: an extended wear electrode patch including a battery, an electrocardiography (ECG) patch input, and a Direct Current (DC) analog front end, the DC analog front end including: a first op-amp having a first input port, a second input port, and a first output port; and a second op-amp having a third input port, a fourth input port, and a second output port; wherein: the battery is coupled between the ECG patch input and the first input port; the ECG patch input is in communication with the first input port; the first output port is in communication with the fourth input port; and the second output port is in communication with the ECG patch input.

Clause 19. The wearable electrocardiography monitor of clause 18, wherein the DC analog front end includes: a common voltage source; a third op-amp having a fifth input port, a sixth input port, and a third output port, wherein: the second output port is in communication with the first input port; the third output port is in communication with the second input port; the fifth input port is in communication with the common voltage source; and the sixth input port is in communication with the second output port.

Clause 20. The wearable electrocardiography monitor of clause 18, wherein the DC analog front end includes: a common voltage source; a third op-amp having a fifth input port, a sixth input port, and a third output port, wherein: the second output port is in communication with the third input port and the third output port; the fifth input port is in communication with the common voltage source; and the third output port is in communication with the first input port and the sixth input port.

Certain terms are used throughout the description and claims to refer to particular features or components. As one skilled in the art will appreciate, different persons may refer to the same feature or component by different names. This document does not intend to distinguish between components or features that differ in name but not function.

As used herein, various units of measure may be referred to by associated short forms with various prefixes applied thereto as set by the International System of Units (SI), which one of ordinary skill in the relevant art will be familiar with.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the present description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

While the invention has been particularly shown and described as referenced to the embodiments thereof, those skilled in the art will understand that the foregoing and other changes in form and detail may be made therein without departing from the spirit and scope.

What is claimed is:

1. A Direct Current (DC) coupled analog front end for an electrocardiogram (ECG) measuring device, comprising:
an amplifying stage including a first op-amp that is configured to receive a signal input from an ECG patch at a first input port and a feedback signal at a second input port and to produce an amplified output of an ECG signal via a first output port;
a feedback stage including a second op-amp that is configured to receive the amplified output and produce the feedback signal at a second output port; and
a DC source, coupled between the signal input from the ECG patch and the first input port.

2. The DC coupled analog front end of claim 1, further comprising a passive lowpass filter in communication between the first output port of the first op-amp and a signal output for an ECG signal.

3. The DC coupled analog front end of claim 1, wherein the second op-amp is an inverting op-amp that receives the amplified output at an inverting input port and a common voltage at a feedback input port to produce the feedback signal, wherein an input resistor is disposed between the inverting input port and the first output port of the first op-amp and an input capacitor is disposed between the inverting input port and the second output port.

4. The DC coupled analog front end of claim 3, further comprising:
a third op-amp disposed in the feedback stage, and configured to produce a second feedback signal that the first op-amp is configured to receive at the second input port with the feedback signal produced by the second op-amp received at the first input port of the first op-amp, the third op-amp receiving the feedback signal at a second inverting import port and the common voltage at a second feedback input port to produce the second feedback signal.

5. The DC coupled analog front end of claim 1, further comprising a third op-amp disposed in the feedback stage, wherein:
the second op-amp provides the feedback signal to the second input port of the first op-amp and receives the amplified output at a first feedback input port; and
the third op-amp provides a second feedback signal to the first input port of the first op-amp and to a second feedback input port of the third op-amp.

6. The DC coupled analog front end of claim 5, further comprising a common voltage source wherein a second feedback signal is provided to the common voltage source and the feedback signal is provided to the second input port of the first op-amp.

7. The DC coupled analog front end of claim 1, further comprising:
a feedback resistor and a feedback capacitor arranged in series between the feedback stage and the second input port of the first op-amp.

8. The DC coupled analog front end of claim 1, further comprising:
a resistive feedback loop, connected between the first output port of the first op-amp and the second input port of the first op-amp that provides the amplified output as at least a portion of the feedback signal.

9. The DC coupled analog front end of claim 1, further comprising:
a capacitive feedback loop, connected between the first output port of the first op-amp and the second input port of the first op-amp that provides the amplified output as at least a portion of the feedback signal.

10. The DC coupled analog front end of claim 1, wherein the DC source has a voltage of 2.5 Volts.

11. A Direct Current (DC) coupled analog front end for an electrocardiogram (ECG) measuring device, comprising:
an ECG patch input;
a first op-amp having a first input port, a second input port, and a first output port;
a second op-amp having a third input port, a fourth input port, and a second output port; and
a DC source, wherein:
the DC source is coupled between the ECG patch input and the first input port;
the ECG patch input is in communication with the first input port;
the first output port is in communication with the fourth input port.

12. The DC coupled analog front end of claim 11, further comprising:
an ECG signal output; and
a passive lowpass filter,
wherein the passive lowpass filter in communication between the first output port and the signal output.

13. The DC coupled analog front end of claim 11, further comprising:
an input resistor; and
an input capacitor,
wherein:
the second op-amp is an inverting op-amp;
the fourth input port is an inverting input port;
the third port is in communication with a common voltage source;
the input resistor is disposed between the first output port and the fourth input port; and
the input capacitor is disposed between the third input port and the second output port.

14. The DC coupled analog front end of claim 13, further comprising:
a third op-amp, having a fifth input port, a sixth input port, and a third output port;
wherein:
the second output port is in communication with the first input port;
the third output port is in communication with the second input port;
the fifth input port is in communication with the common voltage source; and
the sixth input port is in communication with the second output port.

15. The DC coupled analog front end of claim 11, further comprising:
a common voltage source; and
a third op-amp having a fifth input port, a sixth input port, and a third output port;
wherein:
the second output port is in communication with the third input port and the third output port;
the fifth input port is in communication with the common voltage source;
the third output port is in communication with the first input port and the sixth input port.

16. The DC coupled analog front end of claim 15, wherein:
the third output port is in communication with a negative terminal of the DC source; and
the first output port is in communication with the second input port.

17. The DC coupled analog front end of claim 1, further comprising:
a feedback resistor and a feedback capacitor arranged in series between the second output port and the second input port.

18. A wearable electrocardiography monitor, comprising:
an extended wear electrode patch including a battery, an electrocardiography (ECG) patch input, and a Direct Current (DC) analog front end, the DC analog front end including:
a first op-amp having a first input port, a second input port, and a first output port; and
a second op-amp having a third input port, a fourth input port, and a second output port;
wherein:
the battery is coupled between the ECG patch input and the first input port;
the ECG patch input is in communication with the first input port;
the first output port is in communication with the fourth input port; and
the second output port is in communication with the ECG patch input.

19. The wearable electrocardiography monitor of claim 18, wherein the DC analog front end includes:
a common voltage source;
a third op-amp having a fifth input port, a sixth input port, and a third output port, wherein:
the second output port is in communication with the first input port;
the third output port is in communication with the second input port;
the fifth input port is in communication with the common voltage source; and
the sixth input port is in communication with the second output port.

20. The wearable electrocardiography monitor of claim 18, wherein the DC analog front end includes:
a common voltage source;
a third op-amp having a fifth input port, a sixth input port, and a third output port, wherein:
the second output port is in communication with the third input port and the third output port;
the fifth input port is in communication with the common voltage source; and
the third output port is in communication with the first input port and the sixth input port.
